(19) United States
(12) Patent Application Publication
(10) Pub. No.: US 2004/0258832 A1
(43) Pub. Date: Dec. 23, 2004

(54) Method of chemical analysis using microwells patterned from self-assembled monolayers and substrates

(76) Inventors: Anna M. Barklund, Kirkland, WA (US); Dan L. Jin, Kirkland, WA (US); Jeffrey K. Kressbach, Snobhomish, WA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/463,174

(22) Filed: Jun. 17, 2003

(51) Int. Cl. B05D 3/00; C12Q 1/68; C12M 1/34

(52) U.S. Cl. 427/2.11; 435/6; 435/287.2

(57) ABSTRACT

A method for chemical analysis is provided comprising using a substrate that includes a hydrophilic silicon oxide surface and a hydrophobic self-assembled silane monolayer overlying and covalently bonded to portions of the silicon oxide surface to form a pattern defining a plurality of microwells, forming a solution comprising at least one chemical analyte in at least one of the microwells, and performing a chemical analysis on the solution.

### BACKGROUND OF THE INVENTION

All patents, patent applications, and publications cited within this application are incorporated herein by reference to the same extent as if each individual patent, patent application or publication was specifically and individually incorporated by reference.

The invention relates generally to chemical analysis methods using a multi-well sampling apparatus to achieve high throughput identification of chemicals, chemical libraries, and chemical mixtures. These types of analyses are particularly useful in assays and screenings of high volumes of chemical compounds in the pharmaceutical, biotechnology, and material sciences.

Currently, there is a strong need for high throughput identification of chemical compounds in pharmaceutical sciences since methods such as combinatorial chemistry can generate millions of potentially pharmaceutical candidates. In the fields of biotechnology and genomics, partial digestions of proteins and DNA yield many different polypeptides or short, single stranded DNA that often desirably are identified. The chemical compounds can be identified by any method that exploits unique spectroscopic, photochemical, biochemical, bioelectronic, immunochemical, electrical, optical, or chemical properties of a specific compound. In some cases, such as immunochemical assays, a target compound is identified through interaction with another chemical compounds (e.g., antigen-antibody interactions). The analytical methods used are application specific and can include high performance liquid chromatography, mass spectrometry, nuclear magnetic resonance spectroscopy, ultraviolet spectroscopy, visible spectroscopy, infrared spectroscopy, or fluorescence spectroscopy.

Many methods and instruments have been developed to perform multi-well, high throughput chemical analysis including liquid dispensers and spotters for microarrays, specialized detector interfaces, microarray readers, and microarray environment controllers, for example see, U.S. Pat. Nos. 6,569,385; 6,558,623; 6,548,171; 6,545,758; 6,489,106; 6,485,918; 6,482,593; 6,448,089; 6,447,723; 6,101,946; and 5,922,617. In general, the wells are arrayed in rows and columns to give a number of wells, for example 4 rows×6 columns (24 wells) or 8 rows×12 columns (96 wells). Small well sizes are crucial to realize dense arrays on reasonably sized substrates, and well sizes on the order of hundreds of microns (i.e., microwells) are desirable. However, the analysis methods can be hindered by the expense and difficulty in producing cleanly defined and chemically stable microwells by currently used techniques. In some cases, molding a polymer pattern on a surface produces microwells that are microns deep. In other cases, the microwells are formed by positive or negative tone photolithography, and other chemical processes sometimes are needed to stabilize the patterned photoresist. However, such microwells can be eroded by chemicals used in the analysis, can suffer physical damage during sampling by the instrument, or can peel off the substrate due to adhesion problems induced by chemicals or heating/cooling cycles. Thus, there is currently a need in the field for chemical analysis methods that do not use microwells comprising chemically molded polymers of patterned, chemically stabilized photoresists.

### SUMMARY OF THE INVENTION

A method for chemical analysis is provided comprising using a substrate that includes a hydrophilic silicon oxide surface and a hydrophobic self-assembled silane monolayer overlying and covalently bonded to portions of the silicon oxide surface to form a pattern defining a plurality of microwells, forming a solution comprising at least one chemical analyte in at least one of the microwells, and performing a chemical analysis on the solution. Self-assembled monolayers (“SAMs”) are monolayers formed of molecules each having a functional group that selectively attaches to a particular surface, with the remainder of each molecule interacting with neighboring molecules in the monolayer to form a relatively ordered array. With this method, problems encountered during high throughput chemical analysis associated with lack of adhesion of the microwells to the substrate are avoided since the microwells are patterned from silane monolayers that are covalently bonded to the surface of the substrate. In addition, the silane monolayers are robust to solutions, which are usually the medium for analysis of chemicals such as proteins, DNA, and RNA. The method is compatible with any techniques used to identify or analyze chemical compounds or mixtures, including high performance liquid chromatography, gas chromatography, mass spectrometry, nuclear magnetic resonance spectroscopy, ultraviolet spectroscopy, visible spectroscopy, infrared spectroscopy, and fluorescence spectroscopy. In many embodiments, the solutions can be formed individually in each of the microwells or simultaneously in many of the microwells using microliter-dispensing systems. Alternatively, the solutions can be formed in the microwells from bulk solutions through dipping, spraying, or spreading processes. The chemical analyte in the solution can be generated before or after forming the solution in the microwells.

The details of one or more embodiments are set forth in the accompanying drawings and the description below. Other features, objects, and advantages will be apparent from the description and drawings, and from the claims.

### BRIEF DESCRIPTION OF THE DRAWINGS

FIG. 1 is a cross-sectional, magnified illustrative view of the substrate used in the method.

FIG. 2 is an illustrative view of the silane monolayer self-assembled and covalently bonded to the hydrophilic silicon oxide surface of the substrate.

FIG. 3 is an illustrative top view of the substrate showing an array of bins formed from the silane monolayer on the hydrophilic silicon oxide surface.

FIG. 4 illustrates possible configurations for polar samples relative to the depth of the wells formed from the silane monolayers.

FIG. 5 illustrates formation of the polar solutions on the substrate from bulk polar solutions.

### DETAILED DESCRIPTION

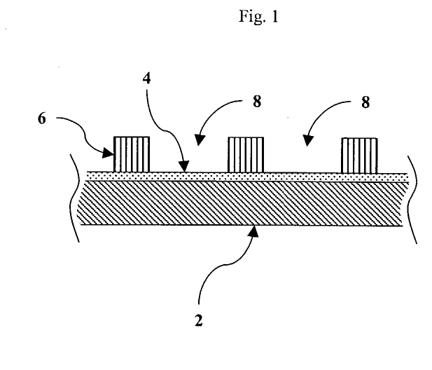

In one embodiment a method for chemical analysis comprises, referring to FIG. 1, providing a substrate (2) having a hydrophilic silicon oxide surface (4), the surface having a pattern comprising a hydrophobic self-assembled silane monolayer (6), wherein the pattern defines a plurality of microwells (8); forming a solution comprising at least one chemical analyte in at least one of the microwells; and performing a chemical analysis on the solution. The solution should be chosen so that it preferentially interacts with either the hydrophilic or hydrophobic portions of the substrate. When the solution is hydrophilic (i.e., a polar solvent), the solution will be formed over the hydrophilic portions. When the solution is hydrophobic (i.e., a nonpolar solvent), the solution will be formed over the hydrophobic portions. Preferably, the solution is an aqueous solution. Preferably, forming the solution comprising at least one chemical analyte includes dispensing a predetermined volume of solution comprising at least one chemical analyte on the substrate so that the predetermined volume is in at least one of the microwells. More preferably, forming the solution comprising at least one chemical analyte includes dispensing a plurality of predetermined volumes of solutions simultaneously on the substrate so that the predetermined volumes are in a plurality of microwells, the solutions each comprising at least one chemical analyte.

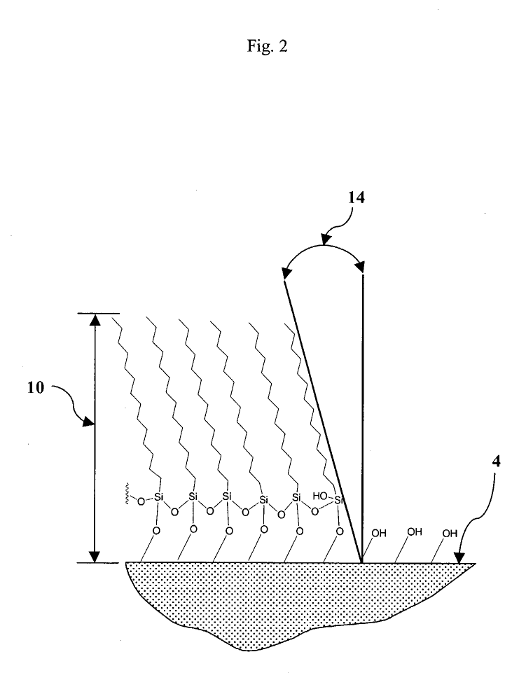

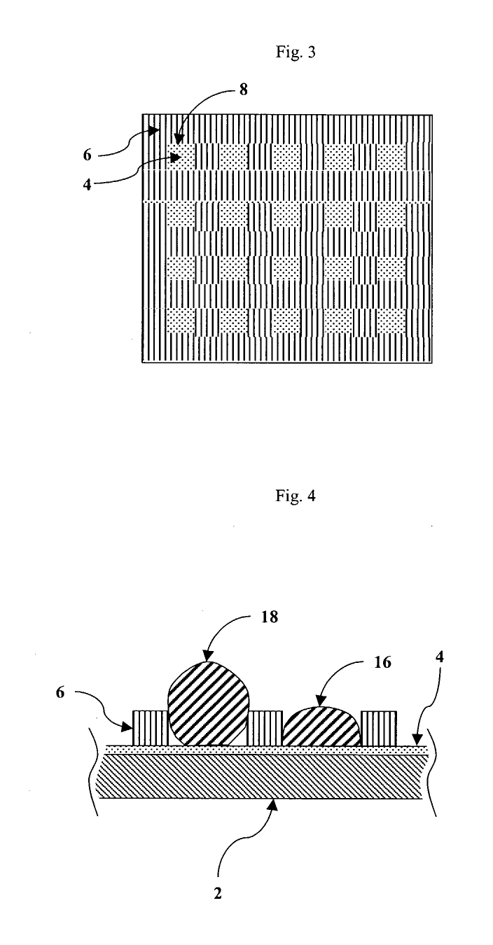

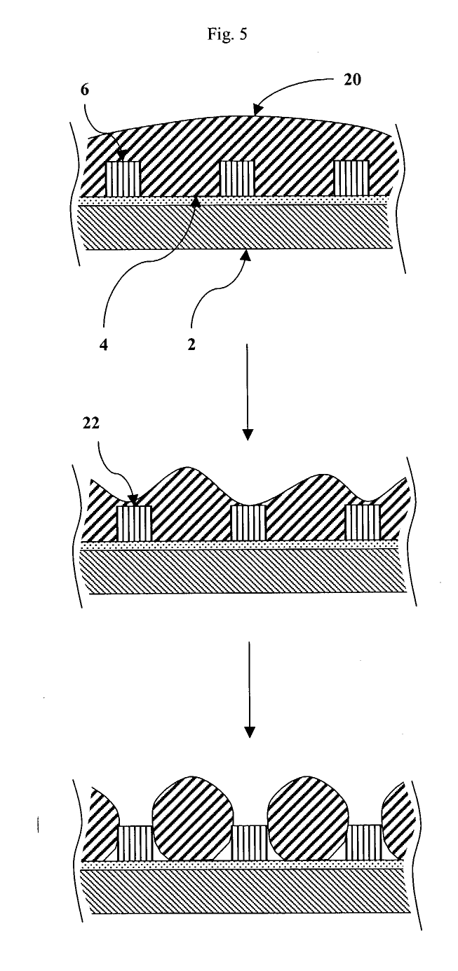

The thickness of the hydrophobic self-assembled silane monolayer is typically from about 10 Angstroms to about 50 Angstroms. Referring to FIG. 2, the thickness (10) of the monolayer depends on the length of the alkyl group (12) on the silane and the angle (14) made by the alkyl group normal to the hydrophilic silicon oxide surface (4). When the solution comprising at least one chemical analyte is an aqueous solution, the physiochemical repulsion of the water by the hydrophobic monolayer confines the aqueous solution comprising the chemical analyte to the hydrophilic surface. Referring to FIG. 3, which is a top view of the substrate, the microwells (8) are defined by the hydrophilic silicon oxide surface (4) bordered by the hydrophobic self-assembled silane monolayer (6). Referring to FIG. 4 for examples where the solution comprising at least one chemical analyte interacts preferably with the hydrophilic portions, the volume of solution can extend beyond the thickness (18) of the silane monolayer and need not be confined (16) to the thickness of the silane monolayer (6) since there is sufficiently low surface tension (i.e., repulsion) between the polar solution and hydrophobic self-assembled monolayer relative to the high surface tension between the polar solution and hydrophilic surface (4). Preferably, microwells are arranged in rows and columns.

In another embodiment of the method, forming the solution comprising at least one chemical analyte includes a) contacting an area of the substrate containing two or more microwells with the solution comprising at least one chemical analyte and b) allowing the solution comprising at least one chemical analyte to segregate into two or more microwells. In this embodiment, the solution comprising at least one chemical analyte may also be referred to as a “bulk solution.” The segregation of the bulk solution into the microwells results from the repulsive interaction of the between the solution and either the hydrophobic or hydrophilic portions. For example, when the solution is polar, forming the solution in the microwells from a bulk solution is shown in FIG. 5, where a bulk solution (20) is contacted with area of the substrate (2) containing two or more microwells, the solution begins segregating due to the low surface tension (22) between the polar solution and the hydrophobic self-assembled monolayer, and finally the polar solution fully segregates into two or more of the microwells. The bulk solution may be applied to the substrate by a variety of methods including dipping the substrate into a bulk solution and removing the substrate from the bulk solution, condensing the solution from the gas phase to the liquid phase on the substrate, spraying the solution on the substrate, or spreading the solution across the substrate.

In some embodiments, forming the solution of chemical analyte includes a) depositing a precursor solution in at least one microwell and b) activating the precursor solution to provide the solution comprising at least one chemical analyte. In these embodiments, the precursor solution may comprise a chemical different from the chemical analyte or a chemical that is the same as the chemical analyte. For example, the precursor solution may comprise the chemical analyte at concentrations below the detection limits of a particular analysis method, thereby requiring amplification of the chemical analyte in order to increase the concentration (e.g., the precursor chemical is DNA that is amplified to a higher concentration using a polymerase chain reaction (PCR) to give a chemical analyte). An example of a method where the precursor solution may comprise a different chemical from the chemical analyte is when the precursor chemical is mixed with another chemical to form a covalent or noncovalent complex, the covalent or noncovalent complex being the chemical analyte (e.g., when a protein is complexed with a molecule in order to detect the molecule-protein complex). Such an approach may be particularly useful, for example, in screening chemical compounds or libraries where pharmaceutical candidates are identified by their ability to bind certain proteins (e.g., antigen-antibody complexes). Depositing the precursor solution can include dispensing a predetermined volume of precursor solution on the substrate so that the predetermined volume is in at least one of the microwells. Preferably, depositing the precursor solution includes dispensing plurality of predetermined volumes of precursor solutions simultaneously on the substrate so that the predetermined volumes are in a plurality of microwells. In another embodiment, depositing the precursor solution includes a) contacting an area of the substrate containing two or more microwells with the precursor solution and b) allowing the precursor solution to segregate into one or more microwells.

In many embodiments, the silane portion of the hydrophobic self-assembled monolayer comprises from about 12 to about 24 carbons. Preferably, the hydrophobic self-assembled monolayer contains fluorine. Silane monolayers can be formed by covalent reactions between alkyl halosilanes and hydroxy groups on the silicon oxide surface, producing hydrogen chloride (HCl) as a by-product. The halosilanes can contain one, two, or three halogens, where chlorine is the preferred halogen. The self-assembled silane monolayer can be deposited by techniques known to those skilled in the art such as spin coating, dip coating, brushing, spreading, or spraying. When the self-assembled silane monolayer is deposited on substrate using a solution of halosilane, it is preferable that the solvent is chemically inert toward the halosilane (e.g., dry toluene, dry hexane, eic.).

The dimensions of the patterned microwells are measured along the hydrophilic silicon oxide surface to the edge of the silane monolayers. The surface area of the exposed silicon oxide surface constituting each microwell preferably ranges from about 9 μm² to about 40,000 μm². This surface area may be achieved using microwells having dimensions ranging from about 3 μm×3 μm to about 400 μm to about 400 μm. However, the microwells do not need to square, but also can be rectangular, oval, or a combination thereof. The microwells can be patterned using photolithographic techniques with positive or negative tone photoresists.

The hydrophilic silicon oxide surface can be deposited on a substrate or can be used as the substrate by itself. A substrate that does not have a hydrophilic silicon oxide surface must be compatible with deposition of a hydrophilic silicon oxide surface or transformable through chemical reaction into a hydrophilic silicon oxide surface. The hydrophilic silicon oxide surface can be chemical vapor deposited (CVD) SiO₂. In other embodiments, the hydrophilic silicon oxide substrate can be quartz or glass. Preferably, the substrate is silicon and the silicon oxide surface comprises SiO₂ with a thickness of about 4 μm to about 6 μm.

In preferred embodiments, the chemical analyte comprises a protein, a deoxyribonucleic acid (DNA), or a ribonucleic acid (RNA). The protein, DNA, or RNA may also contain other molecular moieties such as conjugated fluorescent probes, mass spectrometry tags, radiological probes, or complexed chemical substrates. “Probes” or “tags” are chemical moieties that have a known response in certain chemical analysis methods. For example, fluorescent probes have a known fluorescent wavelength and mass spectrometry tags have known ion fragmentation pattern. The chemical analysis can be any method that detects by spectroscopic, photochemical, biochemical, bioelectronic, immunochemical, electrical, optical, or chemical means. Preferably, the chemical analysis is high performance liquid chromatography, gas chromatography, mass spectrometry, ultraviolet spectroscopy, visible spectroscopy, infrared spectroscopy, or fluorescence spectroscopy.

The patterned substrate can be prepared using photolithographic, spin coating, and dry etching techniques. In one example, a 6-inch silicon wafer with a 6 μm silicon oxide surface was used. The wafer was: 1) cleaned with an ammonium hydroxide/hydrogen peroxide solution; 2) dump/rinsed in 18 Meg/ohm DI water several times; 3) spin/rinse dried with 18 Meg/ohm DI water; 4) spin coated on the silicon oxide surface with Futurex™ negative photoresist to give a thickness of 1.0 μm and baked at 150° C. for 60 seconds; 5) aligned with a photomask such that the eventual hydrophobic areas are defined by the chrome and the eventual hydrophilic areas are defined by the clear field; 6) exposed to 365 nm UV light, 35 mW/cm² for 10.0 seconds in hard contact with the photomask; 7) developed in Shipley MF-24A TMAH developer until the pattern cleared; 8) dump/rinsed in 18 Meg/ohm DI water; 9) spin/rinse dried with 18 Meg/ohm DI water; 10) plasma etched in O₂ for 3 minutes at 200 watts; 11) spin coated on the silicon oxide surface by application of a 0.2% solution of 1H, 1H, 2H, 2H perfluoroundecyl trichlorosilane in toluene at 500 rpm followed by additional spinning at 1000 rpm for 5 seconds, flushing with toluene, and finally ramping to 3000 rpm and spinning for 30 seconds; 12) baked in air on hot plate at 100° C. for 60 seconds; 13) resist stripped in a solvent stripper to give the pattern of hydrophobic silane monolayer on the hydrophilic surface; 14) rinsed in isopropyl alcohol; 15) dump rinsed in 18 Meg/ohm DI water; and 16) spin/rinse dried with 18 Meg/ohm DI water.

In another example, a 6-inch silicon wafer with a silicon oxide surface was: 1) cleaned in an ammonium hydroxide/hydrogen peroxide solution; 2) dump/rinsed in 18 Meg/ohm DI water; 3) spin/rinse dried with 18 Meg/ohm DI water; 4) spin coated on the silicon oxide surface by application of a 0.2% solution of 1H, 1H, 2H, 2H perfluoroundecyl trichlorosilane in toluene at 500 rpm followed by additional spinning at 1000 rpm for 5 seconds, flushing with toluene, and finally ramping to 3000 rpm and spinning for 30 seconds; 5) baked in air on hot plate at 150° C. for 60 seconds; 6) spin coated with a positive photoresist to give a thickness of 1.2 μm; 7) soft baked at 110° C. for 90 seconds; 8) aligned with a photomask such that the eventual hydrophobic areas are defined by the chrome and the eventual hydrophilic areas are defined by the clear field; 9) exposed to 365 nm UV light, 35 mW/cm² for 3.5 seconds; 10) developed with Shipley MF-24A TMAH developer until the pattern cleared; 11) dump rinsed in 18 Meg/ohm DI water; 12) spin/rinse dried with 18 Meg/ohm DI water; 13) DRIE etched; 14) resist stripped to give the hydrophobic/hydrophilic pattern; 15) rinsed in isopropyl alcohol; 16) dump rinsed in 18 Meg/ohm DI water; and 17) spin/rinse dried with 18 Meg/ohm DI water.

Other embodiments are within the following claims.

1. A method, comprising: a) providing a substrate that includes a hydrophilic silicon oxide surface and a hydrophobic self-assembled silane monolayer overlying and covalently bonded to portions of the silicon oxide surface to form a pattern defining a plurality of microwells; b) forming a solution comprising at least one chemical analyte in at least one of the microwells; and c) performing a chemical analysis on the chemical analyte.

2. The method of claim 1, wherein forming the solution comprising at least one chemical analyte includes dispensing a predetermined volume of solution comprising at least one chemical analyte on the substrate so that the predetermined volume is in at least one of the microwells.

3. The method of claim 1, wherein forming the solution comprising at least one chemical analyte includes dispensing a plurality of predetermined volumes of solutions simultaneously on the substrate so that the predetermined volumes are in a plurality of microwells, the solutions each comprising at least one chemical analyte.

4. The method of claim 1, wherein forming the solution comprising at least one chemical analyte includes a) contacting an area of the substrate containing two or more microwells with the solution comprising at least one chemical analyte and b) allowing the solution comprising at least one chemical analyte to segregate into one or more microwells.

5. The method of claim 1, wherein forming the solution of chemical analyte includes a) depositing a precursor solution comprising at least one precursor chemical in at least one microwell and b) activating the precursor chemical to provide the chemical analyte, thereby forming the solution comprising at least one chemical analyte.

6. The method of claim 5, wherein depositing the precursor solution includes dispensing a predetermined volume of precursor solution comprising at least one analyte on the substrate so that the predetermined volume is in at least one of the microwells.

7. The method of claim 5, wherein depositing the precursor solution includes dispensing a plurality of predetermined volumes of precursor solutions simultaneously on the substrate so that the predetermined volumes are in a plurality of microwells.

8. The method of claim 5, wherein depositing the precursor solution includes a) contacting an area of the substrate containing two or more microwells with the precursor solution and b) allowing the precursor solution to segregate into one or more microwells.

9. The method of claim 1, wherein the solution is an aqueous solution.

10. The method of claim 1, wherein the silane of the hydrophobic self-assembled silane monolayer comprises from about 12 to about 24 carbons.

11. The method of claim 10, wherein the hydrophobic self-assembled monolayer comprises fluorine.

12. The method of claim 1, wherein the microwells are arranged in rows and columns.

13. The method of claim 1, wherein each microwell comprises an area of the silicon oxide surface ranging in size from about 9 μm² to about 40,000 m.²

14. The method of claim 13, wherein each microwell has dimensions ranging from about 3 μm×3 μm to about 200 μm×200 μm.

15. The method of claim 1, wherein the silicon oxide substrate is chemical vapor deposited SiO₂, quartz, or glass.

16. The method of claim 1, wherein the substrate is silicon and the silicon oxide surface comprises SiO₂ with a thickness of about 4 to about 6 μm.

17. The method of claim 1, wherein the chemical analyte is selected from the group consisting of proteins, deoxyribonucleic acids, ribonucleic acids, and combinations thereof.

18. The method of claim 1, wherein the chemical analysis is selected from the group consisting of high performance liquid chromatography, gas chromatography, mass spectrometry, nuclear magnetic resonance spectroscopy, ultraviolet spectroscopy, visible spectroscopy, infrared spectroscopy, fluorescence spectroscopy, and combinations thereof.
